(19) United States
(12) Patent Application Publication
(10) Pub. No.: US 2025/0291324 A1
(43) Pub. Date: Sep. 18, 2025

(54) MACHINE LEARNING FORCE FIELDS MODEL TRAINED WITH OFF-EQUILIBRIUM FORCE FIELD DATA

(71) Applicant: Microsoft Technology Licensing, LLC, Redmond, WA (US)

(72) Inventors: Han YANG, Shanghai (CN); Jielan LI, Shanghai (CN); Ziheng LU, Shanghai (CN); Hongxia HAO, Shanghai (CN)

(73) Assignee: Microsoft Technology Licensing, LLC, Redmond, WA (US)

(21) Appl. No.: 19/211,947

(22) Filed: May 19, 2025

Related U.S. Application Data
(63) Continuation of application No. PCT/CN2024/081751, filed on Mar. 14, 2024.

(51) Int. Cl. G05B 13/04; G05B 13/02; G16C 10/00

(52) U.S. Cl. CPC G05B 13/048; G05B 13/0265; G16C 10/00

(57) ABSTRACT

A computing system including one or more processing devices configured to obtain sets of ground-state force field data associated with equilibrium chemical systems. The one or more processing devices are further configured to compute ground-state uncertainty values of the sets of ground-state force field data and select a first subset of the equilibrium chemical systems that have respective ground-state uncertainty values above a ground-state uncertainty threshold. The one or more processing devices are further configured to compute off-equilibrium chemical systems by modifying a respective temperature and/or pressure of each equilibrium chemical system included in the first subset. The one or more processing devices are further configured to compute respective ab initio simulations of a second subset of the off-equilibrium chemical systems to obtain off-equilibrium force field data. The one or more processing devices are further configured to train a machine learning force fields model with the off-equilibrium force field data.

### CROSS REFERENCE TO RELATED APPLICATIONS

This application is a continuation of International Patent Application Serial Number PCT/CN2024/081751, filed Mar. 14, 2024, the entirety of which is hereby incorporated herein by reference for all purposes.

### BACKGROUND

Material design is central to technological advancements in fields such as nanoelectronics, energy storage, biomedicine, and environmental sustainability. Conventionally, the development of new materials has been a slow and expensive process, dominated by experimental trial and error. Transitioning these efforts to a computational environment-referred to as in silico-offers immense potential to expedite this process. In silico material design relies on the ability to simulate the behaviors of materials and predict their properties with sufficient efficiency and accuracy.

### SUMMARY

According to one aspect of the present disclosure, a computing system is provided, including one or more processing devices configured to, during a training phase, obtain sets of ground-state force field data associated with a respective plurality of equilibrium chemical systems. The one or more processing devices are further configured to compute respective ground-state uncertainty values of the sets of ground-state force field data. The one or more processing devices are further configured to select a first subset of the plurality of equilibrium chemical systems. The equilibrium chemical systems included in the first subset have respective ground-state uncertainty values above a ground-state uncertainty threshold. The one or more processing devices are further configured to compute a plurality of off-equilibrium chemical systems at least in part by modifying a respective temperature and/or pressure of each equilibrium chemical system included in the first subset. The one or more processing devices are further configured to compute respective ab initio simulations of a second subset of the plurality of off-equilibrium chemical systems to thereby obtain a plurality of sets of off-equilibrium force field data. The one or more processing devices are further configured to train a machine learning force fields (MLFF) model at least in part with the sets of off-equilibrium force field data.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 schematically shows a computing system during a training phase in which a machine learning force fields (MLFF) model is trained at one or more processing devices, according to one example embodiment.

FIG. 2 schematically shows the computing system in additional detail when the one or more processing devices are configured to receive and process a plurality of equilibrium chemical systems, according to the example of FIG. 1.

FIG. 3 schematically shows the computing system when the one or more processing devices are configured to further process a first subset of the plurality of equilibrium chemical systems, according to the example of FIG. 2.

FIG. 4 schematically shows the computing system when the one or more processing devices are configured to further process a second subset including a plurality of off-equilibrium chemical systems, according to the example of FIG. 3.

FIG. 5 schematically shows the computing system when the MLFF model trained during the training phase is included in a model ensemble, according to the example of FIG. 3.

FIG. 6 schematically shows additional computing processes that may be performed at an uncertainty module in some examples when computing the first subset, according to the example of FIG. 2.

FIG. 7 schematically shows the computing system during an inferencing phase performed subsequently to training the MLFF model, according to the example of FIG. 1.

FIG. 8 shows an example formation energy plot for europium phosphide compounds, according to the example of FIG. 7.

FIG. 9A shows a labeled periodic table indicating an element distribution of a MatterSim training corpus, according to the example of FIG. 1.

FIG. 9B shows a labeled periodic table indicating an element distribution of an MPF2021 training corpus, according to the example of FIG. 1.

FIG. 10A shows a labeled periodic table in which each element is labeled with a corresponding number of identified structures located on or below a current convex hull, according to the example of FIG. 7.

FIG. 10B shows a labeled periodic table in which each element is labeled with a corresponding number of identified structures determined to be stable on a combined convex hull, according to the example of FIG. 10A.

FIG. 10C shows a labeled periodic table in which each element is labeled with a corresponding number of identified structures determined to be stable on a combined convex hull after removal of anion corrections, according to the example of FIG. 10B.

FIG. 11A shows a flowchart of a method for use with a computing system during a training phase of an MLFF model, according to the example of FIG. 1.

FIGS. 11B-11D show additional steps of the method of FIG. 11A that may be performed in some examples.

FIGS. 12A-12D show additional steps of the method of FIG. 11A performed during an inferencing phase subsequently to the training phase.

FIG. 13 shows a schematic view of an example computing environment in which the computing system of FIG. 1 may be instantiated.

### DETAILED DESCRIPTION

Machine learning, and deep learning in particular, has revolutionized the field of materials emulation and property prediction. To simulate materials at the atomic level, machine learning force fields (MLFFs) models have been developed. These MLFF models predict the quantum-chemical relationship between atomic coordinates and the resulting energies and forces. Thus, MLFF models enable the simulation of the dynamics of atomic systems with unprecedented efficiency, allowing material properties to be estimated on a larger scale.

The task of predicting material properties under realistic conditions has been a formidable challenge for previous MLFF models. Existing MLFF models are trained on near-equilibrium-position databases of chemical systems and are therefore inaccurate when predicting the properties of materials under most real-world temperature and pressure conditions. Thus, existing MLFF models are frequently unable to realistically simulate the behaviors of materials in the settings in which those materials would be used.

In order to address the above challenges, the MatterSim MLFF model is provided, as described below. The MatterSim MLFF model is trained to emulate materials dynamics and predict material properties under realistic finite temperature and pressure conditions beyond the near-equilibrium domain. The training dataset used to train MatterSim is generated using an off-equilibrium module that, as discussed in further detail below, samples simulated chemical systems at temperature and pressure values that are selected according to their associated uncertainty values. This sampling approach results in a training corpus with which the MatterSim MLFF model can be trained with high sample efficiency. Force field data is then computed at MatterSim during inferencing time and can be further used to compute material properties such as system stability and phonon dispersion. MatterSim therefore allows for efficient computation of such material properties over a wider range of temperature and pressure conditions relative to previous MLFF models.

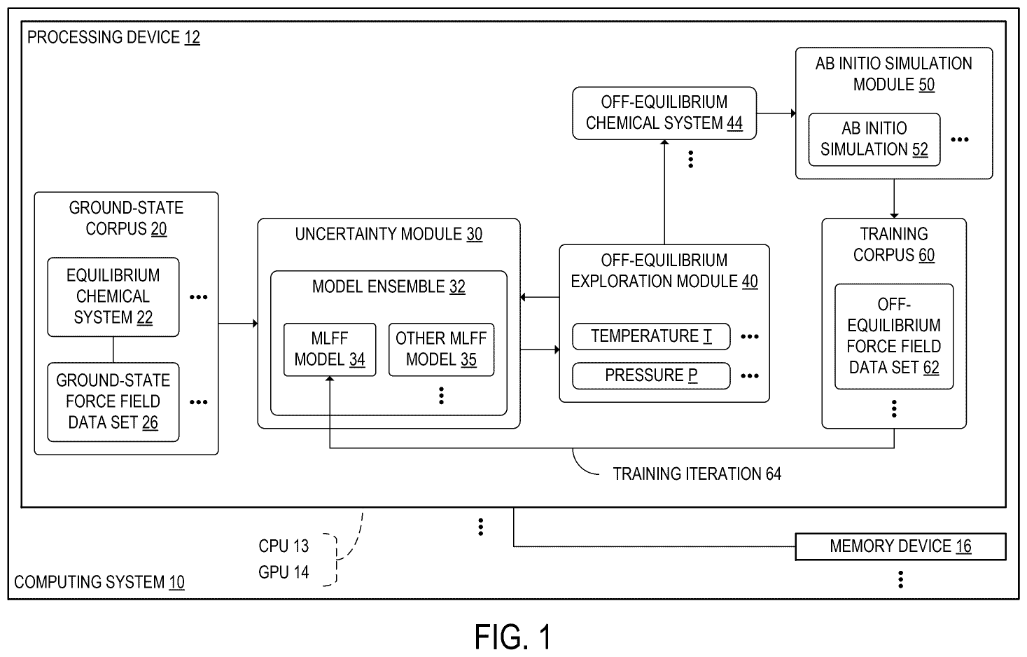

FIG. 1 schematically shows a computing system 10 during a training phase in which the MatterSim MLFF model is trained, according to one example embodiment. The computing system 10 includes one or more processing devices 12, which may, for example, include one or more central processing units (CPUs) 13, one or more graphics processing units (GPUs) 14, and/or one or more other hardware accelerators. The computing system 10 further includes one or more memory devices 16 that are communicatively coupled to the one or more processing devices 12. The one or more memory devices 16, may include one or more volatile memory devices and/or one or more non-volatile memory devices. The computing system 10 may be instantiated at a single physical computing device or may alternatively be a virtual computing system instantiated across a plurality of interconnected physical computing devices. In some examples, the computing system 10 is instantiated at a plurality of physical computing devices located in a data center.

The one or more processing devices 12 are configured to obtain sets of ground-state force field data 26 associated with a respective plurality of equilibrium chemical systems 22. The sets of ground-state force field data 26, along with their associated equilibrium chemical systems 22, form a ground-state corpus 20. The one or more processing devices 12 are further configured to process the ground-state corpus 20 at an uncertainty module 30, an off-equilibrium exploration module 40, and an ab initio simulation module 50 to compute a training corpus 60. The training corpus 60 includes a plurality of off-equilibrium force field data sets 62. The one or more processing devices 12 are further configured to use the training corpus 60 to train an MLFF model 34 over a plurality of training iterations 64.

For example, the one or more processing devices 12 may obtain a plurality of the ground-state force field data sets 26 from one or more pre-generated databases of force field data. Additionally or alternatively, the one or more processing devices 12 may be configured to synthesize additional ground-state force field data sets 26 at the computing system 10. The ground-state corpus 20 may additionally or alternatively include experimentally measured ground-state force field data.

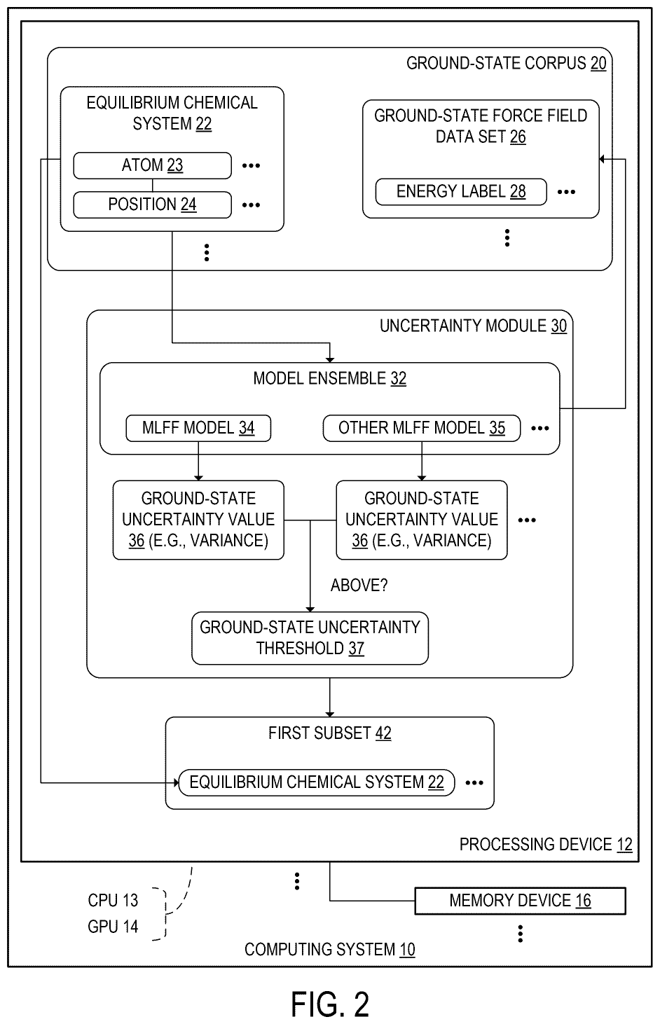

FIG. 2 schematically shows the computing system 10 in additional detail when the one or more processing devices 12 are configured to receive and process the equilibrium chemical systems 22. As shown in the example of FIG. 2, the plurality of equilibrium chemical systems 22 each include a plurality of atoms 23 that have respective positions 24. The atoms 23 in an equilibrium chemical system 22 may, for example, be arranged in a crystal lattice, a metal, a liquid, a gas, a solution of dissolved ions, a covalently bonded molecule, or some other type of chemical system. The respective element of each of the atoms 23 is indicated in the specification of the equilibrium chemical system 22 in which it is included.

The respective ground-state force field data set 26 associated with each equilibrium chemical system 22 includes a plurality of energy labels 28. The energy labels 28 may be potential energy values associated with different points within the equilibrium chemical system 22. Thus, the energy labels 28 form potential energy surfaces within the equilibrium chemical system 22. Additionally or alternatively, the ground-state force field data sets 26 may include force values and/or stress values associated with the atoms 23 included in the equilibrium chemical systems 22.

In the example of FIG. 2, for each of the equilibrium chemical systems 22, the one or more processing devices 12 are configured to input a specification of the equilibrium chemical system 22 into a model ensemble 32 including a plurality of pretrained MLFF models. From the pretrained MLFF models, the one or more processing devices 12 are further configured to obtain a respective plurality of the sets of ground-state force field data 26 that are included in the ground-state corpus 20. In this example, the MLFF model 34 that is trained during the training phase is included in the model ensemble 32. In addition, the model ensemble 32 includes a plurality of other MLFF models 35. The other MLFF models 35 are pretrained models in this example.

The one or more processing devices 12 are further configured to execute an uncertainty module 30. At the uncertainty module 30, as shown in FIG. 2, the one or more processing devices 12 are further configured to compute respective ground-state uncertainty values 36 of the sets of ground-state force field data 26. For each of the equilibrium chemical systems 22, the one or more processing devices 12 may be configured to compute the ground-state uncertainty value 36 for that equilibrium chemical system 22 at least in part by computing a respective variance over the sets of ground-state force field data 26 computed for that equilibrium chemical system 22 at the model ensemble 32. For example, the following equation may be used to compute the ground-state uncertainty values:

$$unc = max [ \frac{1}{N} {\sum }_{i = 1}^{N} {( \vert {F}_{i a} \vert - \vert {\overline{F}}_{a} \vert )}^{2} ]$$

In the above equation, N is the total number of pretrained MLFF models. F̅ₐ is the mean, over the MLFF models included in the model ensemble 32, of atomic force values for the ath atom computed at the different MLFF models. Fᵢₐ is the atomic force value computed at the ith MLFF model for the ath atom. In other examples, the ground-state uncertainty values 36 may instead be computed as maximum variances over potential energy or stress values computed at the MLFF models.

Subsequently to computing the ground-state uncertainty values 36, the one or more processing devices 12 are further configured to select a first subset 42 of the plurality of equilibrium chemical systems 22. The equilibrium chemical systems 22 included in the first subset 42 have respective ground-state uncertainty values 36 that are above a ground-state uncertainty threshold 37. Thus, when the one or more processing devices 12 select the first subset 42, the one or more processing devices 12 select the equilibrium chemical systems 22 for which the outputs of the MLFF models disagree with each other. The one or more processing devices 12 are accordingly configured to narrow the overall set of equilibrium chemical systems 22 to obtain a first subset 42 in which the equilibrium chemical systems 22 are likely to be simulated inaccurately. By performing further simulations of such chemical systems, as discussed in further detail below, the one or more processing devices 12 may obtain training data that is more informative when used to train the MLFF model 34. Selecting the first subset 42 may accordingly increase the sample efficiency of such training.

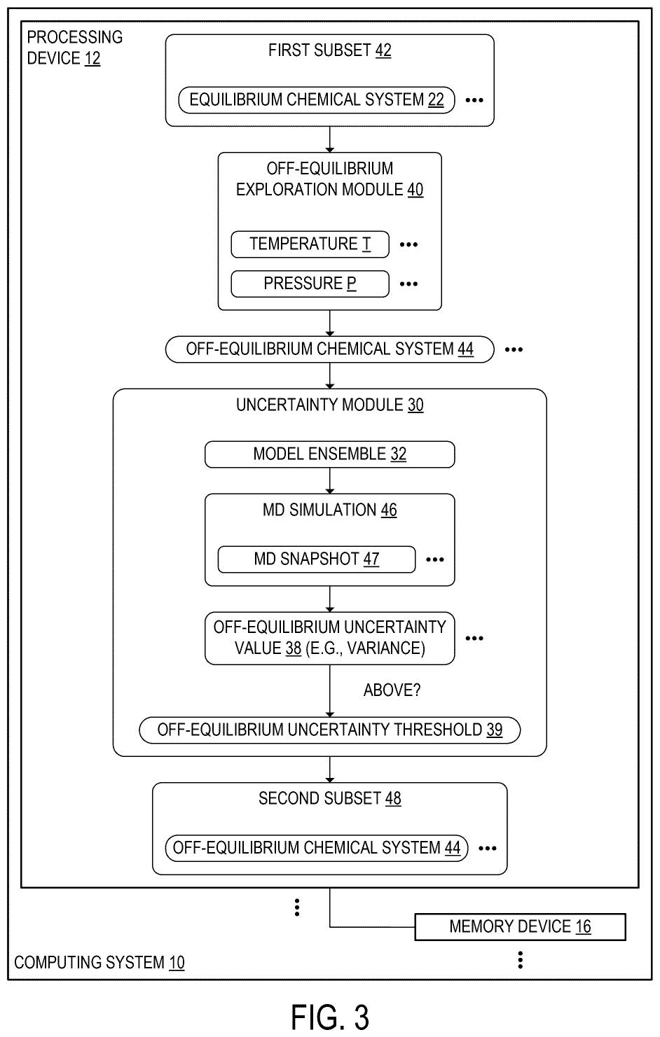

FIG. 3 schematically shows the computing system 10 when the one or more processing devices 12 are configured to further process the first subset 42 of the plurality of equilibrium chemical systems 22. The one or more processing devices 12 are configured to execute the off-equilibrium simulation module 40 to compute a plurality of off-equilibrium chemical systems 44. The off-equilibrium chemical systems 44 are computed at least in part by modifying a respective temperature T and/or pressure P of each equilibrium chemical system 22 included in the first subset 42. The off-equilibrium simulation module 40 is configured to compute a respective plurality of the off-equilibrium chemical systems 44 with different temperature and/or pressure values for each of the equilibrium chemical systems 22. For example, the values of the temperature T may be included in a range from 0-5000 K, and the values of the pressure P may be included in a range from 0-4000 GPa.

As shown in the example of FIG. 3, the one or more processing devices 12 are further configured to compute a second subset 48 of the plurality of off-equilibrium chemical systems 44 as discussed below. When the second subset 48 is computed, the one or more processing devices 12 may be further configured to input specifications of the off-equilibrium chemical systems 44 into the uncertainty module 30 for processing at the model ensemble 32. At the MLFF models included in the model ensemble 32, the one or more processing devices 12 may be further configured to compute a respective plurality of molecular dynamics (MD) simulations 46 of the off-equilibrium chemical systems 44. The MD simulations 46 each include a plurality of MD snapshots 47, which may specify the positions and energy labels of the atoms included in the off-equilibrium chemical systems 44 at respective points in time.

The one or more processing devices 12, in the example of FIG. 3, are further configured to compute a plurality of off-equilibrium uncertainty values 38 of the off-equilibrium chemical systems 44. The off-equilibrium uncertainty values 38 are computed as uncertainty values of the MD snapshots 47 in the example of FIG. 3. Each of the off-equilibrium uncertainty values 38, similarly to each of the ground-state uncertainty values 36, may be computed at least in part by computing a variance over the outputs of the MLFF models included in the model ensemble 32.

The one or more processing devices 12 are further configured to select, as the second subset 48, a plurality of the off-equilibrium chemical systems 44 that have respective off-equilibrium uncertainty values 38 above an off-equilibrium uncertainty threshold 39. Accordingly, the second subset 48 includes off-equilibrium chemical systems 44 for which the MLFF models included in the model ensemble 32 output MD simulations 46 that disagree with each other. Such off-equilibrium chemical systems 44 may accordingly be more informative than the other off-equilibrium chemical systems 44 during training of the MLFF model 34. The sample efficiency of the training data used to train the MLFF model 34 may therefore be further increased by narrowing the plurality of off-equilibrium chemical systems 44 down to the second subset 48.

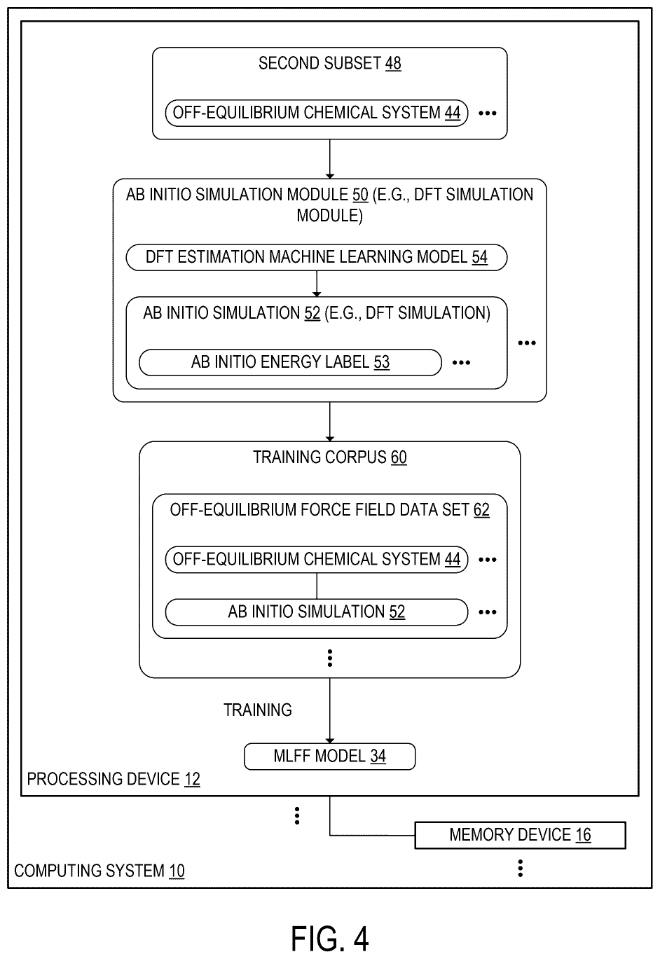

FIG. 4 schematically shows the computing system 10 when the one or more processing devices 12 are configured to further process the second subset 48 of the plurality of off-equilibrium chemical systems 44. At the ab initio simulation module 50, the one or more processing devices 12 are further configured to compute respective ab initio simulations 52 of the second subset 48 of the plurality of off-equilibrium chemical systems 44. Each ab initio simulation 52 includes a plurality of ab initio energy labels 53 that are associated with respective locations within the corresponding off-equilibrium chemical system 44. In the example of FIG. 4, the ab initio simulation module 50 is a density functional theory (DFT) simulation module at which the one or more processing devices 12 are configured to compute the ab initio simulations 52 as DFT simulations.

In some examples, rather than explicitly computing the DFT simulations, the one or more processing devices 12 may be configured to compute the DFT simulations at a DFT estimation machine learning model 54. Since explicit DFT simulation is computationally slow, computing the DFT simulations at a DFT estimation machine learning model 54 may result in significant increases in the speed at which the DFT simulations are generated.

By computing the ab initio simulations 52, the one or more processing devices 12 are configured to obtain a plurality of sets of off-equilibrium force field data 62. Each of the off-equilibrium force field data sets 62 includes an off-equilibrium chemical system 44 included in the second subset 48, along with the associated ab initio energy labels 53 computed at the ab initio simulation module 50.

The plurality of off-equilibrium force field data sets 62 form the training corpus 60. Subsequently to computing the training corpus 60, the one or more processing devices 12 are further configured to train the MLFF model at least in part with the sets of off-equilibrium force field data 62 included in the training corpus 60. Supervised learning may be performed to train the MLFF model 34 in the example of FIG. 4.

The training data generation approach discussed above allows the one or more processing devices 12 to compute a training corpus 60 with which the MLFF model 34 may be trained in a sample-efficient manner. By computing the first subset 42 and the second subset 48, the search space of chemical systems is narrowed in two phases prior to performing the ab initio simulations. In each of these narrowing phases, the one or more processing devices 12 are configured to select simulation results that provide comparatively large amounts of Bayesian evidence. The ab initio simulations 52 that are included in the training corpus 60 are accordingly more informative than other simulation data that could potentially have been used to train the MLFF model 34. Since the overall search space of compositions, temperatures, and pressures is extremely large, the dataset narrowing discussed above selectively samples the search space to produce a training corpus 60 with which the MLFF model 34 can be trained in a practically achievable amount of time.

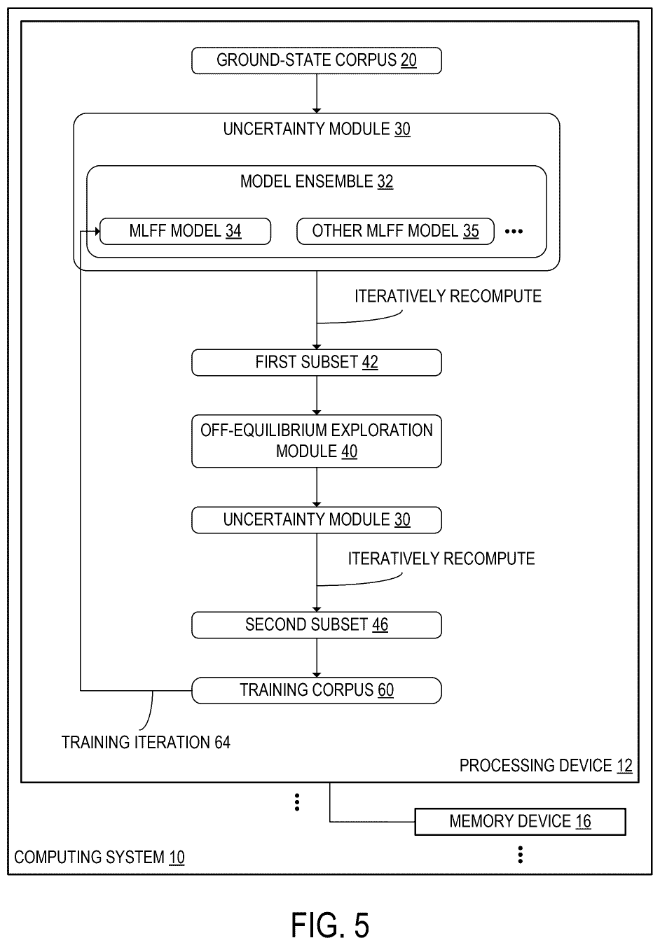

FIG. 5 schematically shows the computing system 10 when a training iteration 64 is performed, according to an example in which the MLFF model 34 trained during the training phase is included in the model ensemble 32. Since the MLFF model 34 is updated over the course of training, the uncertainty computations performed at the uncertainty module 30 also differ. In the example of FIG. 5, the one or more processing devices 12 are further configured to iteratively recompute the first subset 42 of the plurality of equilibrium chemical systems 22 during training of the MLFF model 34. The one or more processing devices 12 may also be configured to iteratively recompute the second subset 48 of the plurality of off-equilibrium chemical systems 44, as shown in the example of FIG. 5. The first subset 42 and the second subset 48 may, for example, be recomputed after a predetermined number of training iterations 64 have been performed. Thus, the MLFF model 34 may be trained via active learning such that the ab initio simulations 52 with which the MLFF model 34 is trained are those associated with off-equilibrium chemical systems 44 that are simulated with high uncertainty, given a current partially trained state of the MLFF model 34.

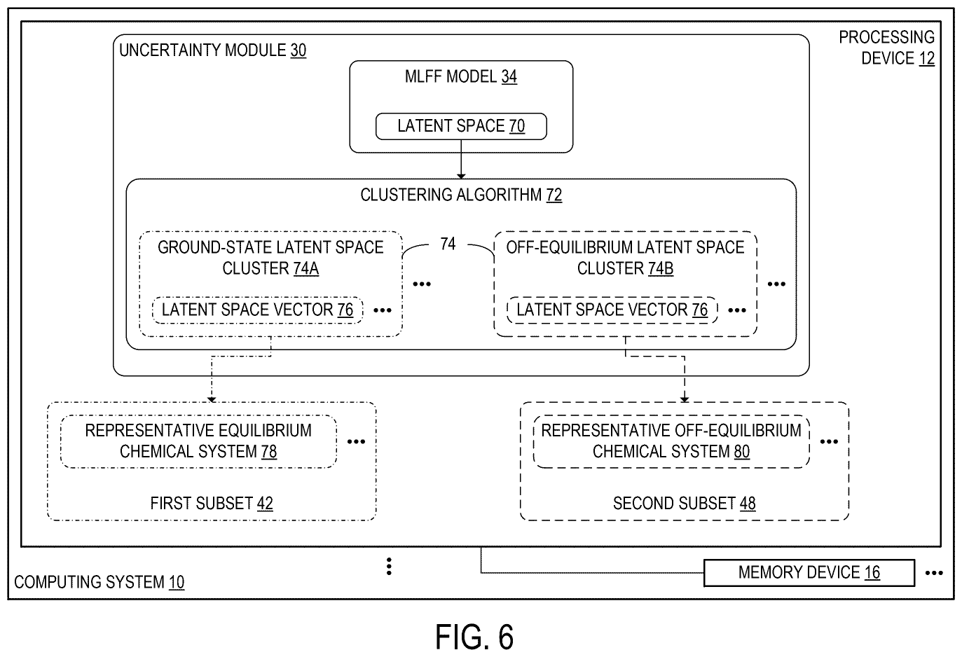

FIG. 6 schematically shows additional computing processes that may be performed at the uncertainty module 30 in some examples when computing the first subset 42. According to the example of FIG. 6, the one or more processing devices 12 may be further configured to compute a plurality of latent space clusters 74 of a latent space 70 of the MLFF model 34. The latent space clusters 74 each include a respective plurality of latent space vectors 76 computed at the MLFF model 34 from the specifications of the equilibrium chemical systems 22. The one or more processing devices 12 may be configured to compute the latent space clusters 74 at a clustering algorithm 72. For example, the clustering algorithm 72 may be the t-Distributed Stochastic Neighbor Embedding (t-SNE) algorithm.

The one or more processing devices 12 may be further configured to select the first subset 42 of the plurality of equilibrium chemical systems 22 at least in part by selecting a plurality of representative equilibrium chemical systems 78 from the latent space clusters 74. For example, the one or more processing devices 12 may be configured to sample a predefined number of representative equilibrium chemical systems 78 from each latent space cluster 74. In some examples, the one or more processing devices 12 are configured to identify a plurality of ground-state latent space clusters 74A that correspond to equilibrium chemical systems and a plurality of off-equilibrium latent space clusters 74B that correspond to off-equilibrium chemical systems. In such examples, the one or more processing devices 12 are further configured to sample the ground-state latent space clusters 74A when selecting the first subset 42.

In some examples, the sampling of representative equilibrium chemical systems 78 shown in FIG. 6 may be performed instead of the uncertainty determination performed in the example of FIG. 3. In other examples, both uncertainty-based sampling and cluster-based sampling may be performed to select the first subset 42. For example, the one or more processing devices 12 may be configured to perform the clustering-based sampling shown in FIG. 6 as a preliminary step of generating the first subset 42 and may be further configured to compute respective ground-state uncertainty values 36 of the representative equilibrium chemical systems 78.

Additionally or alternatively to performing the cluster-based sampling shown in FIG. 6 to identify the first subset 42, the one or more processing devices 12 may be further configured to sample a plurality of representative off-equilibrium chemical systems 80 from the off-equilibrium latent space clusters 74B when selecting the second subset 48. For example, the one or more processing devices 12 may be configured to sample the representative off-equilibrium chemical systems 80 as inputs to the model ensemble 32 that are then used to compute the off-equilibrium uncertainty values 38. Thus, the one or more processing devices 12 may be configured to perform additional filtering of the plurality of off-equilibrium chemical systems 44 when computing the second subset 48.

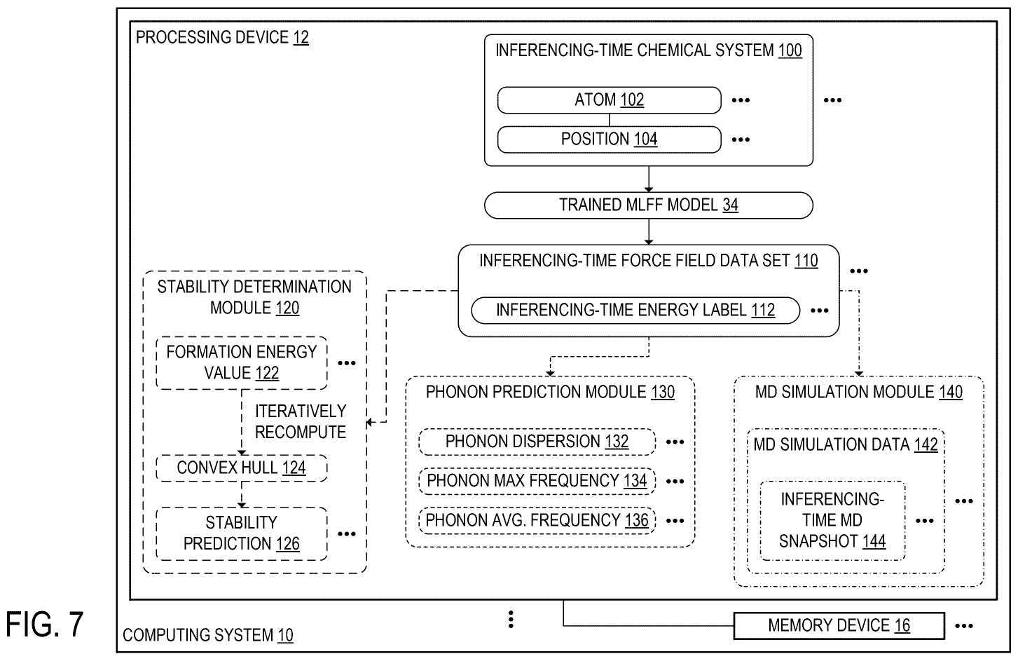

FIG. 7 schematically shows the computing system 10 during an inferencing phase performed subsequently to training the MLFF model 34. In some examples, the training phase and the inferencing phase may be performed at separate computing systems. At the trained MLFF model 34, the one or more processing devices 12 are further configured to compute a set of inferencing-time force field data 110 respectively associated with an inferencing-time chemical system 100. The inferencing-time chemical system 100 includes a plurality of atoms 102 along with the respective positions 104 of those atoms 102. The inferencing-time force field data set 110 includes a plurality of inferencing-time energy labels 112.

The one or more processing devices 12 are further configured to output the set of inferencing-time force field data 110 to an additional computing process. In some examples, as shown in FIG. 7, the additional computing process may be a stability determination module 120. Additionally or alternatively, the one or more processing devices 12 may be configured to output the inferencing-time force field data 110 to a phonon prediction module 130 and/or a molecular dynamics simulation module 140.

At the stability determination module 120, the one or more processing devices 12 are further configured to predict whether the inferencing-time chemical system 100 is stable based at least in part on the inferencing-time force field data 110. The one or more processing devices 12 are further configured to output a stability prediction 126 that indicates whether the inferencing-time chemical system 100 is stable.

In the example of FIG. 7, the one or more processing devices 12 are configured to determine whether a plurality of different inferencing-time chemical systems 100 are stable. In this example, the one or more processing devices 12 are configured to predict whether the inferencing-time chemical systems 100 are stable at least in part by estimating respective formation energy values 122 of the inferencing-time chemical systems 100. These formation energy values 122 are estimated from the inferencing-time energy labels 112.

The one or more processing devices 12 are further configured to iteratively recomputing a convex hull 124 of the plurality of formation energy values 122. The convex hull 124 indicates a lower surface of a plot of formation energy values as a function of composition. The one or more processing devices 12 may be further configured to determine that at least one of the inferencing-time chemical systems 100 is stable in response to determining that the at least one inferencing-time chemical system 100 has a respective formation energy value 122 located on or below the convex hull 124.

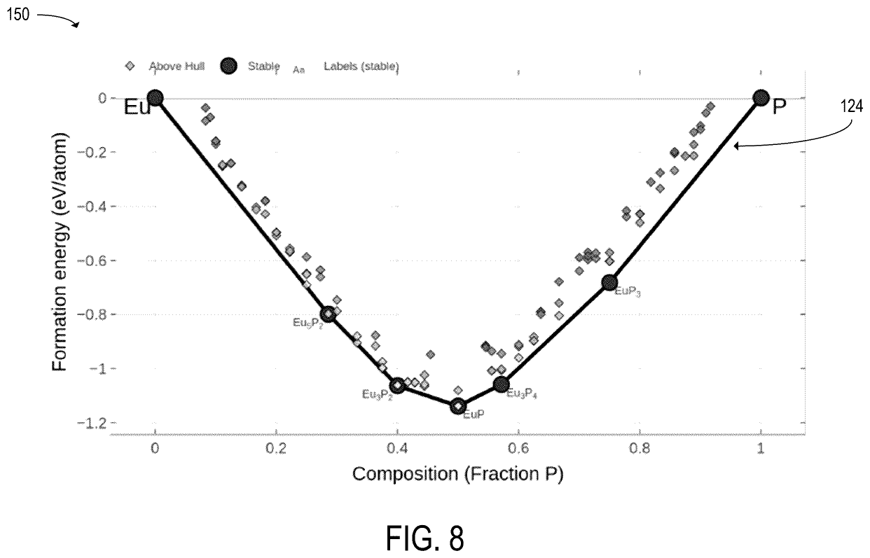

FIG. 8 shows an example formation energy plot 150 for europium phosphide compounds that include different ratios of europium (Eu) to phosphorus (P). Formation energy of different europium phosphide compounds are shown in units of eV/atom as a function of the fraction P in the compound. The convex hull 124 is also shown in the example formation energy plot 150. The stable compounds Eu₅P₂, Eu₃P₂, EuP, Eu₃P₄, and EuP₃ are located on the convex hull 124, along with pure Eu and P. The formation energy plot 150 also shows example configurations that are located above the convex hull 124 and are therefore determined to be unstable.

Returning to the example of FIG. 7, at the phonon prediction module 130, the one or more processing devices 12 may be further configured to predict a phonon dispersion 132 in the inferencing-time chemical system 100 based at least in part on the inferencing-time force field data 110. The one or more processing devices 12 may be further configured to output the phonon dispersion 132.

In some examples, the one or more processing devices 12 are additionally or alternatively configured to compute a phonon average frequency 134 and/or a phonon maximum frequency 136 at the phonon prediction module 130 based at least in part on the inferencing-time force field data 110. The phonon average frequency is defined as follows:

$$\overline{ω} = \frac{\int ω g ( ω ) d ω}{\int g ( ω ) d ω}$$

where ω is phonon angular frequency and g(ω) is the phonon density of states.

The above phonon-related predictions provide an alternative to ab initio phonon dispersion prediction techniques such as the frozen-phonon (FP) approach and density functional perturbation theory (DFPT), which tend to be computationally expensive. Thus, the MLFF model 34 may be more practical to use when performing materials discovery via large-scale materials screening.

In some examples, based at least in part on the inferencing-time force field data 110, the one or more processing devices 12 are further configured to compute MD simulation data 142 of the inferencing-time chemical system 100 at the MD simulation module 140. The MD simulation data 142 includes a plurality of inferencing-time MD snapshots 144 of the inferencing-time chemical system 100 at respective timesteps. The one or more processing devices 12 are further configured to output the MD simulation data 142. By performing molecular dynamics simulations during the inferencing phase, the one or more processing devices 12 are accordingly configured to simulate the movement of the atoms 102 included in the inferencing-time chemical system 100.

Experiments performed by the inventors using the MatterSim MLFF model are discussed below. In these experiments, the MatterSim MLFF model was trained by performing additional training on a pretrained Graphormer v2 model using a training corpus 60 generated with the methods discussed above. The training corpus 60 that was generated in these experiments included over 3 million points of off-equilibrium force field data corresponding to temperatures ranging from 0-5000 K and to pressures ranging from 0-4000 GPa. The ground-state corpus 20 that was used to generate this training corpus included equilibrium chemical systems 22 and ground-state force field data sets 26 obtained from databases including Materials Project Force 2021 (MPF2021), Alexandria, and the Open Quantum Materials Database (OQMD), along with equilibrium chemical systems 22 and ground-state force field data sets 26 generated with Random Structure Search (RSS) and with the diffusion model MatterGen. The ab initio simulations 52 were DFT simulations computed using the Vienna Ab initio Simulation Package (VASP).

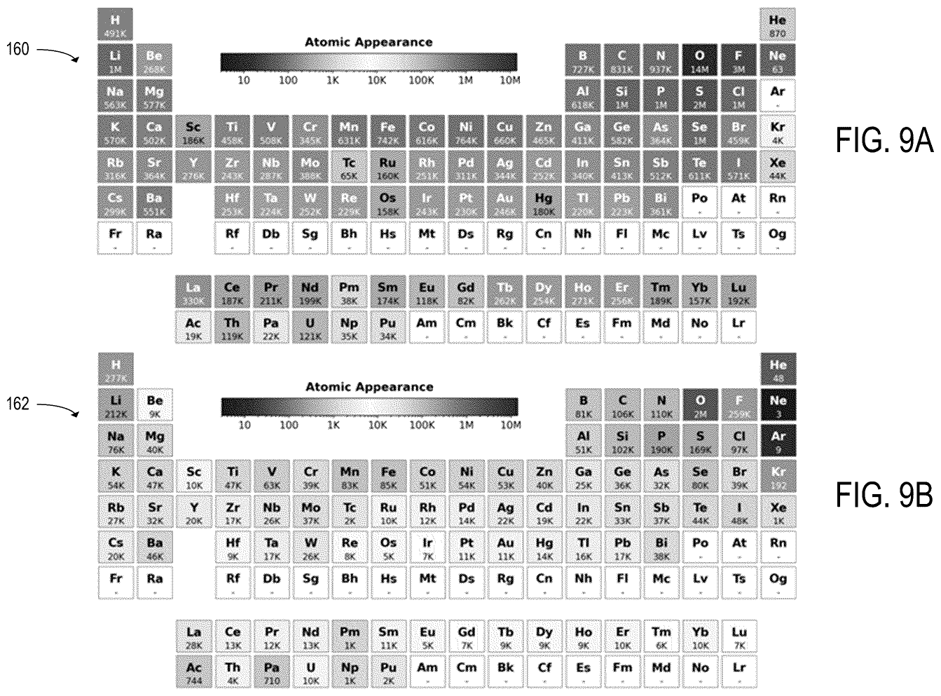

FIGS. 9A-9B respectively show the element distribution of the MatterSim training corpus and the preexisting MPF2021 training corpus. FIG. 9A shows a labeled periodic table 160 in which each element is labeled with the approximate number of atoms of that element that occur in the training corpus 60 of MatterSim. FIG. 9B shows a labeled periodic table 162 in which each element is labeled with the approximate number of atoms of that element that occur in the MPF2021 training corpus. As shown in FIGS. 9A-9B, the MatterSim training corpus is significantly larger than the MPF2021, with approximately ten times the number of atoms of most elements compared to MPF2021.

In one of the experiments the inventors performed, the trained MatterSim MLFF model was used to perform materials discovery by making stability predictions 126 for inferencing-time chemical systems 100. The performance of the MatterSim MLFF model was evaluated on the Matbench Discovery benchmark. The following table summarizes the performance of MatterSim compared to several other MLFF models on Matbench Discovery:

| Model | F1 | Precision | Accuracy | TPR | TNR | MAE | RMSE | R² |
|---|---|---|---|---|---|---|---|---|
| MatterSim | 0.82 | 0.83 | 0.94 | 0.81 | 0.97 | 0.03 | 0.07 | 0.84 |
| CHGNet | 0.58 | 0.52 | 0.84 | 0.66 | 0.88 | 0.07 | 0.11 | 0.61 |
| M3GNet | 0.57 | 0.45 | 0.80 | 0.77 | 0.81 | 0.07 | 0.11 | 0.60 |
| MACE | 0.57 | 0.47 | 0.81 | 0.72 | 0.83 | 0.07 | 0.11 | 0.63 |
| ALIGNN | 0.56 | 0.50 | 0.83 | 0.65 | 0.87 | 0.09 | 0.15 | 0.27 |
| MEGNet | 0.51 | 0.46 | 0.81 | 0.57 | 0.86 | 0.13 | 0.20 | −0.28 |
| CGCNN | 0.51 | 0.45 | 0.81 | 0.59 | 0.85 | 0.14 | 0.23 | −0.62 |
| CGCNN + P | 0.51 | 0.41 | 0.78 | 0.67 | 0.80 | 0.11 | 0.18 | 0.03 |
| Wrenformer | 0.48 | 0.36 | 0.74 | 0.69 | 0.75 | 0.10 | 0.18 | −0.04 |
| BOWSR | 0.44 | 0.32 | 0.68 | 0.74 | 0.67 | 0.12 | 0.16 | 0.14 |
| Voronoi RF | 0.34 | 0.26 | 0.67 | 0.51 | 0.70 | 0.14 | 0.21 | −0.31 |
| Dummy | 0.19 | 0.17 | 0.68 | 0.23 | 0.77 | 0.12 | 0.18 | 0.00 |

As shown in the above table, MatterSim achieves the highest performance among any of the tested models in its F1 score, precision, accuracy, true positive ratio (TPR), true negative ratio (TNR), mean absolute error (MAE), root mean squared error (RMSE), and R² values.

As another materials discovery experiment, an exhaustive search over binary chemical systems (systems including atoms of two different elements) was performed using RSS for 89 different elements. MatterSim was then used to screen the binary chemical systems for stability. 4005 binary element-wise combinations of elements were screened, with 45 different compositions for each combination, resulting in a total of 35,644,500,000 energy inferences. This screening was performed using MatterSim in approximately one week, whereas performing this screening using DFT would take approximately 60,000 years.

The exhaustive search performed using RSS and MatterSim identified 492,939 new structures. 16,399 of the new structures were located on or below the currently known convex hull, which was computed using DFT calculations. By combining the 16,399 identified materials with the current convex hull, a combined convex hull was generated, thereby providing a stricter check on the stability of the structures. 2,416 materials were accordingly identified as having stable structures on the combined hull. Following the identification of the 2,416 stable structures, the anion corrections applied to many materials in the Materials Project database were excluded, resulting in a set of 852 discovered stable binary materials.

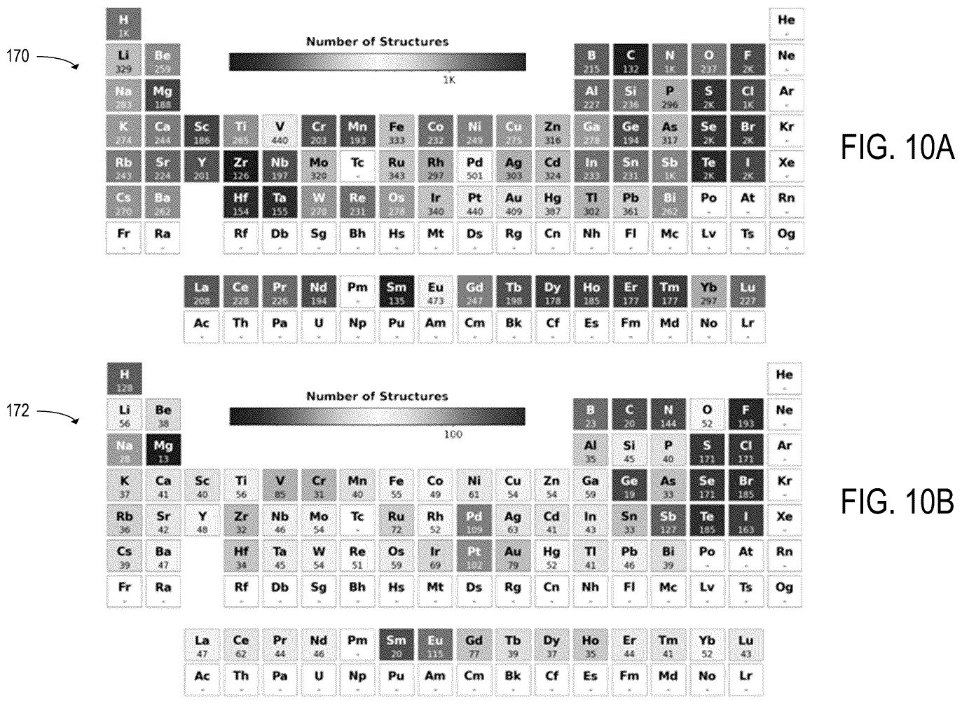

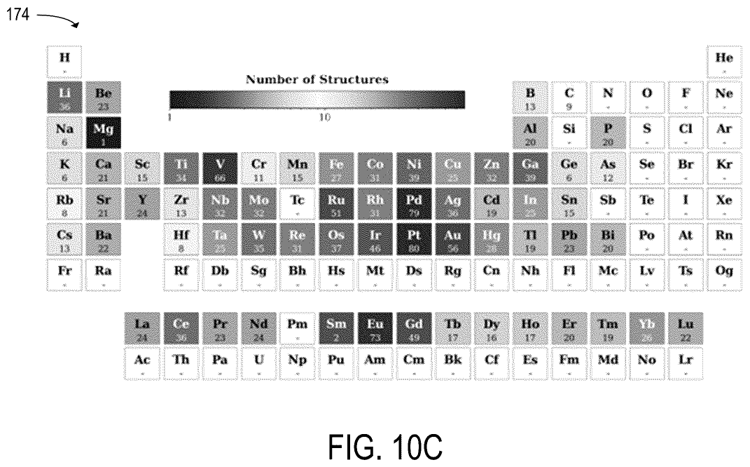

FIGS. 10A-10C show respective labeled periodic tables 170, 172, and 174. In the labeled periodic table 170 of FIG. 10A, each element is labeled with the number of newly identified structures located on or below the current convex hull, of the 16,399 that were identified. In the labeled periodic table 172 of FIG. 10B, each element is labeled with the number of new structures that were determined to be stable on the combined convex hull, of the 2,416 that were identified. In the labeled periodic table 174 of FIG. 10C, each element is labeled with the number of new structures that were located on or below the combined convex hull after removal of the anion corrections. In the labeled periodic table 174 of FIG. 10C, the materials including H, Si, N, Sb, O, S, Se, Te, F, Cl, Br, and I are excluded due to the potential inaccuracy of the anion corrections.

The formation energies of the 852 stable binary materials were used to update the convex hulls of 529 chemical systems, including the Eu—P system discussed above with reference to FIG. 8. In the example of the Eu—P system, the convex hull was updated to include the EuP, Eu₃P₂, and Eu₅P₂ compounds, which were computed as having respective formation energies of −5.83×10⁻² eV/atom, −1.80×10⁻¹ eV/atom, and −1.42×10⁻¹ eV/atom.

The inventors also performed a phonon prediction experiment using MatterSim. In the phonon prediction experiment, phonon dispersions were predicted in 150 different bulk materials. For example, phonon predictions were performed for silicon (Si), gallium nitride (GaN), and perovskite (BaTiO₃). To quantitatively evaluate the phonon predictions made using MatterSim, the phonon maximum frequency and phonon average frequency were computed for each of the simulated phonons. The performance of MatterSim on the phonon prediction task was compared to that of MACE-MP-0. In addition, the phonons predicted using MatterSim were compared to phonon simulations obtained from PhononDB, which were predicted by performing Perdew-Burke-Ernzerhof (PBE) calculations. The MAE and R² values for both MatterSim and MACE-MP-0 were computed for the ML-generated frequencies as functions of the PBE-generated frequencies. The MAE was computed as follows over the density of states:

$${MAE}_{DOS} = \int \vert {g}_{PBE} ( ω ) - {g}_{M L} ( ω ) \vert dω$$

In the above equation, g_{PBE}(ω) and g_{ML}(ω) are the PBE-calculated and ML-predicted phonon densities of states, respectively.

The MAE and R² values of the MatterSim and MACE-MP-0 phonon prediction approaches, as compared to PBE, are summarized in the following table:

| | MAE (max | R² (max | MAE (avg. | R² (avg. |
|---|---|---|---|---|
| Method | frequency) | frequency) | frequency) | frequency) |
| MatterSim | 15.42 | 1.00 | 11.34 | 0.99 |
| MACE-MP-0 | 43.53 | 0.97 | 34.23 | 0.87 |

As shown in the above table, MatterSim outputs more accurate frequency predictions than MACE-MP-0 and almost exactly matches the frequencies obtained from PBE calculations. MatterSim maintained a consistently high level of performance across the prediction of both phonon maximum frequency and phonon average frequency, whereas MACE-MP-0 exhibited a marked decline in R² score on the phonon average frequency prediction task.

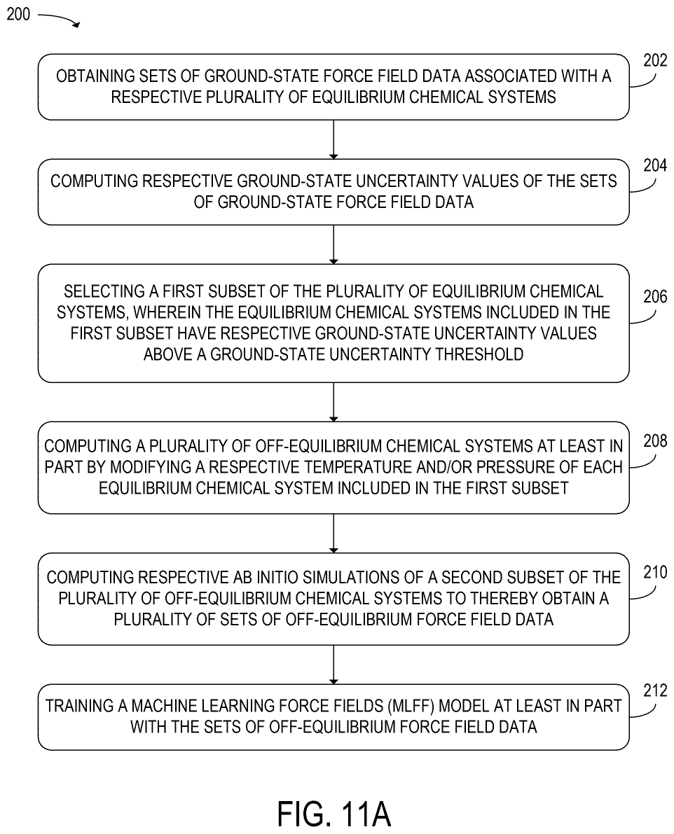

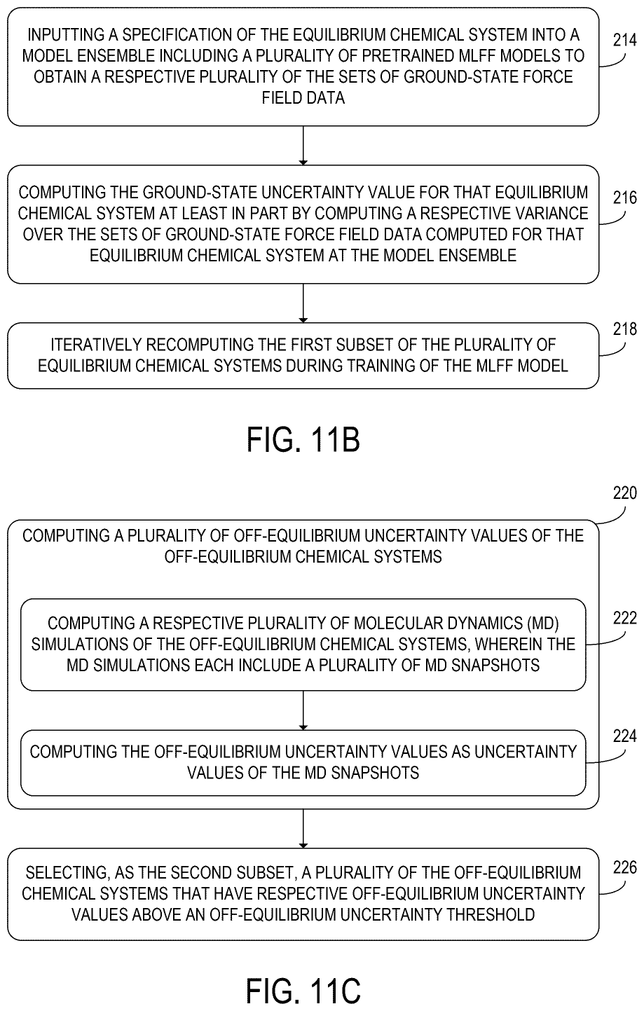

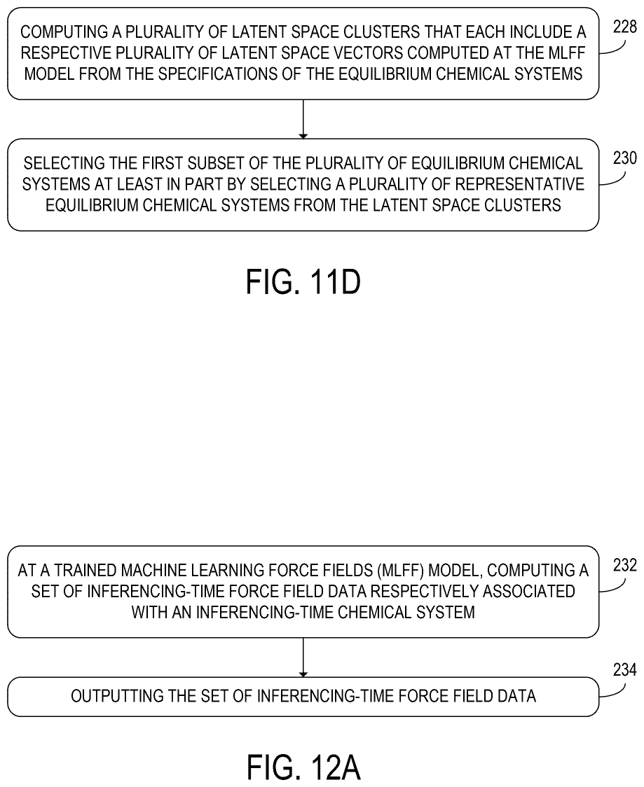

FIG. 11A shows a flowchart of a method 200 for use with a computing system during a training phase of an MLFF model. The method 200 includes, at step 202, obtaining sets of ground-state force field data associated with a respective plurality of equilibrium chemical systems. The equilibrium chemical systems each include a respective plurality of atoms, along with positions indicated for those atoms. The sets of ground-state force field data may each include a plurality of energy labels associated with respective positions in the equilibrium chemical systems. In some examples, the ground-state force field data may additionally or alternatively include force labels and/or stress labels.

At step 204, the method 200 further includes computing respective ground-state uncertainty values of the sets of ground-state force field data. The ground-state uncertainty value of a set of ground-state force field data indicates an extent to which the ground-state force field data is likely to be inaccurate. At step 206, the method 200 further includes selecting a first subset of the plurality of equilibrium chemical systems. The equilibrium chemical systems included in the first subset have respective ground-state uncertainty values above a ground-state uncertainty threshold. Thus, the ground-state corpus that includes the sets of ground-state force field data is narrowed to a first subset that includes the ground-state force field data sets that are estimated to have low accuracy.

At step 208, the method 200 further includes computing a plurality of off-equilibrium chemical systems. The off-equilibrium chemical systems are computed at least in part by modifying a respective temperature and/or pressure of each equilibrium chemical system included in the first subset. A plurality of different off-equilibrium chemical systems may be generated from each of the equilibrium chemical systems in the first subset in order to represent those equilibrium chemical systems at a range of different temperatures and/or pressures.

At step 210, the method 200 further includes computing respective ab initio simulations of a second subset of the plurality of off-equilibrium chemical systems. Thus, a plurality of sets of off-equilibrium force field data are obtained. The second subset may, for example, be selected using an additional set of uncertainty values computed for the off-equilibrium chemical systems. The ab initio simulations may, for example, be DFT simulations. In some such examples, the DFT simulations may be computed at a DFT estimation machine learning model rather than being computed through explicit DFT simulation.

At step 212, the method 200 further includes training an MLFF model at least in part with the sets of off-equilibrium force field data. The MLFF model may be trained via supervised learning. Since the training data is narrowed via the selection of the subsets at steps 206 and 210, the sample efficiency of the off-equilibrium force field data is increased. Thus, the MLFF model may be trained to simulate wide ranges of temperature and pressure values, without consuming impractically large amounts of time or computing hardware.

FIGS. 11B-11C show additional steps of the method 200 of FIG. 10A that may also be performed during the training phase in some examples. Step 214, shown in FIG. 11B, may be performed at step 202 when at least a portion of the ground-state force field data is obtained. At step 214, the method 200 may further include inputting a specification of the equilibrium chemical system into a model ensemble including a plurality of pretrained MLFF models to obtain a respective plurality of the sets of ground-state force field data.

Step 216 may be performed when the ground-state uncertainty values are computed at step 214. At step 216, the method 200 may further include computing the ground-state uncertainty value for that equilibrium chemical system at least in part by computing a respective variance over the sets of ground-state force field data computed for that equilibrium chemical system at the model ensemble. For example, the ground-state uncertainty value computed for an equilibrium chemical system may be a maximum variance among a plurality of different sets of the ground-state force field data computed for that equilibrium chemical system.

In some examples, the MLFF model trained during the training phase is included in the model ensemble. In such examples, at step 218, the method 200 may further include iteratively recomputing the first subset of the plurality of equilibrium chemical systems during training of the MLFF model. Thus, active learning is performed by iteratively refining the ground-state force field data at the MLFF model over the course of training. Iteratively recomputing the first subset may further focus the training of the MLFF model on high-uncertainty chemical systems to thereby further increase the sample efficiency.

FIG. 11C shows additional steps of the method 200 that may be performed to compute the second subset. At step 220, the method 200 may further include computing a plurality of off-equilibrium uncertainty values of the off-equilibrium chemical systems. The off-equilibrium uncertainty values may also be computed using the model ensemble that is used to compute the ground-state uncertainty values in some examples.

At step 222, step 220 may further include computing a respective plurality of MD simulations of the off-equilibrium chemical systems. The MD simulations may each include a plurality of MD snapshots of the off-equilibrium chemical system at corresponding points in time. At step 224, step 220 may further include computing the off-equilibrium uncertainty values as uncertainty values of the MD snapshots. The off-equilibrium uncertainty values may, for example, be computed as respective variances over the sets of MD snapshots computed for each of the off-equilibrium chemical systems.

At step 226, the method 200 may further include selecting, as the second subset, a plurality of the off-equilibrium chemical systems that have respective off-equilibrium uncertainty values above an off-equilibrium uncertainty threshold. Thus, the sample efficiency of training the MLFF model may be increased by narrowing its training corpus to off-equilibrium chemical systems for which the ab initio simulations are likely to provide high predictive value.

FIG. 11D shows additional steps of the method 200 that may be performed in some examples when selecting the equilibrium chemical systems at step 206. At step 228, the method 200 may further include computing a plurality of latent space clusters. The latent space clusters each include a respective plurality of latent space vectors computed at the MLFF model from the specifications of the equilibrium chemical systems. For example, the t-SNE clustering algorithm may be used to compute the latent space clusters.

At step 230, the method 200 may further include selecting the first subset of the plurality of equilibrium chemical systems at least in part by selecting a plurality of representative equilibrium chemical systems from the latent space clusters. For example, a predefined number of representative equilibrium chemical systems indicated by respective latent space vectors may be selected from the clusters. This clustering-based approach to selecting the first subset may allow for representative sampling of the equilibrium chemical systems when the first subset is selected. The uncertainty computation discussed above with reference to step 204 may be performed before or after the latent space cluster sampling. In some examples, latent space sampling may also be performed when selecting the second subset. In such examples, the representative off-equilibrium chemical systems included in the second subset may be sampled from clusters of the latent space corresponding to off-equilibrium chemical systems.

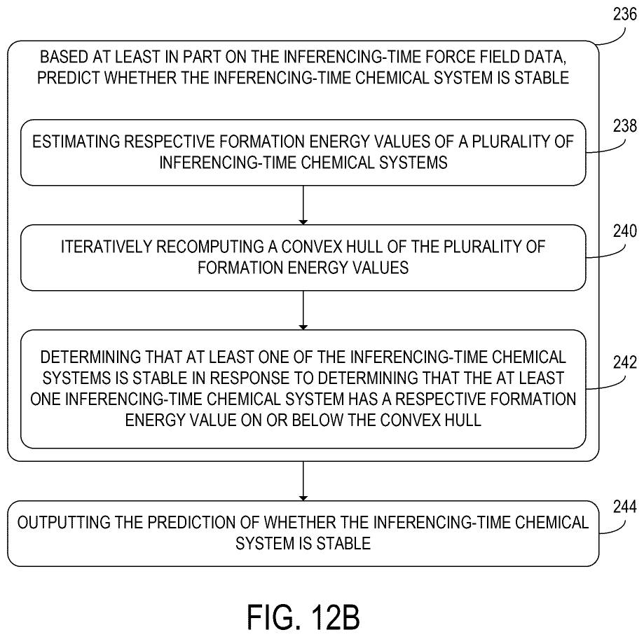

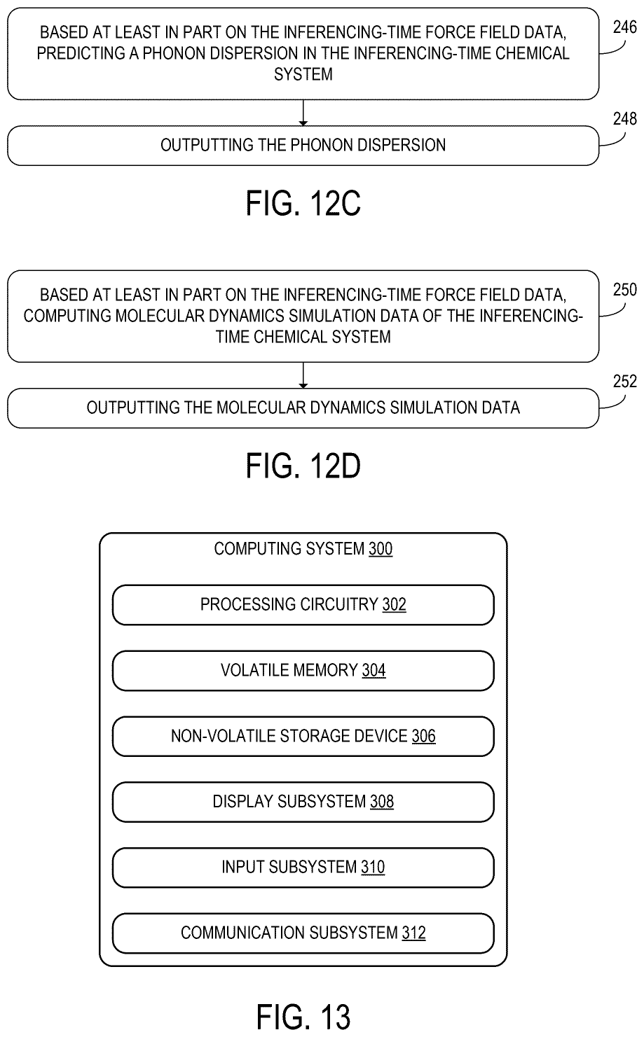

FIGS. 12A-12D show additional steps of the method 200 that are performed during an inferencing phase subsequently to the training phase. The steps of FIGS. 12A-12D may be performed at the same computing system as the steps shown in FIGS. 11A-11D, or may alternatively be performed at one or more other computing systems. At step 232, as shown in FIG. 12A, the method 200 further includes computing a set of inferencing-time force field data at the trained MLFF model. The inferencing-time force field data is associated with an inferencing-time chemical system that is input into the MLFF model. At step 234, the method further includes outputting the set of inferencing-time force field data to an additional computing process.

FIG. 12B shows additional steps of the method 200 that may be performed in examples in which the additional computing process is a stability determination module. At step 236, the method 200 may further include predicting whether the inferencing-time chemical system is stable based at least in part on the inferencing-time force field data. Predicting whether the inferencing-time chemical system is stable at step 236 may include, at step 238, estimating respective formation energy values of a plurality of inferencing-time chemical systems. The plurality of inferencing-time chemical systems includes the inferencing-time chemical system received at step 232.

At step 240, step 236 may further include iteratively recomputing a convex hull of the plurality of formation energy values. The convex hull forms a lower bound on the formation energy values of stable chemical systems. At step 242, the method 200 further includes determining that at least one of the inferencing-time chemical systems is stable in response to determining that the at least one inferencing-time chemical system has a respective formation energy value on or below the convex hull. Thus, such an inferencing-time chemical system may be on or below the lower bound of the current estimate of boundary between stable and unstable configurations.

At step 244, the method 200 may further include outputting the prediction of whether the inferencing-time chemical system is stable. The MLFF model may accordingly be used at inferencing time for the discovery of new, potentially stable materials.

FIG. 12C shows additional steps of the method 200 that may be performed in examples in which the additional computing process is a phonon prediction module. At step 246, the method 200 may further include predicting a phonon dispersion in the inferencing-time chemical system based at least in part on the inferencing-time force field data. At step 248, the method 200 may further include outputting the phonon dispersion. In some examples, a phonon maximum frequency and/or a phonon average frequency is also computed and output at the phonon prediction module.

FIG. 12D shows additional steps of the method 200 that may be performed in examples in which the additional computing process is an MD simulation module. At step 250, the method 200 may further include computing MD simulation data of the inferencing-time chemical system based at least in part on the inferencing-time force field data. The MD simulation data may include a plurality of inferencing-time MD snapshots corresponding to different points in time. At step 252, the method 200 may further include outputting the molecular dynamics simulation data.

Using the systems and methods discussed above, an MLFF model may be trained and executed in a manner that allows chemical systems to be simulated under off-equilibrium conditions. The training dataset of the MLFF model includes ab initio simulations of chemical systems at different temperature and pressure values. Since the search space of possible compositions, temperatures, and pressures is extremely large, the training dataset for the MLFF model is generated in a manner that selects off-equilibrium chemical systems that are likely to be highly informative during MLFF model training. Thus, the MLFF model may be trained with high sample efficiency. Subsequently to training, the MLFF model may be used to simulate a variety of chemical system properties such as stability, phonon dispersion, and molecular dynamics.

In some embodiments, the methods and processes described herein may be tied to a computing system of one or more computing devices. In particular, such methods and processes may be implemented as a computer-application program or service, an application-programming interface (API), a library, and/or other computer-program product.

FIG. 13 schematically shows a non-limiting embodiment of a computing system 300 that can enact one or more of the methods and processes described above. Computing system 300 is shown in simplified form. Computing system 300 may embody the computing system 10 described above and illustrated in FIG. 1. Components of computing system 300 may be included in one or more personal computers, server computers, tablet computers, home-entertainment computers, network computing devices, video game devices, mobile computing devices, mobile communication devices (e.g., smartphone), and/or other computing devices, and wearable computing devices such as smart wristwatches and head mounted augmented reality devices.

Computing system 300 includes processing circuitry 302, volatile memory 304, and a non-volatile storage device 306. Computing system 300 may optionally include a display subsystem 308, input subsystem 310, communication subsystem 312, and/or other components not shown in FIG. 13.

Processing circuitry 302 typically includes one or more logic processors, which are physical devices configured to execute instructions. For example, the logic processors may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors configured to execute software instructions. Additionally or alternatively, the logic processor may include one or more hardware logic circuits or firmware devices configured to execute hardware-implemented logic or firmware instructions. Processors of the processing circuitry 302 may be single-core or multi-core, and the instructions executed thereon may be configured for sequential, parallel, and/or distributed processing. Individual components of the processing circuitry 302 optionally may be distributed among two or more separate devices, which may be remotely located and/or configured for coordinated processing. For example, aspects of the computing system 300 disclosed herein may be virtualized and executed by remotely accessible, networked computing devices configured in a cloud-computing configuration. In such a case, these virtualized aspects are run on different physical logic processors of various different machines. These different physical logic processors of the different machines will be understood to be collectively encompassed by processing circuitry 302.

Non-volatile storage device 306 includes one or more physical devices configured to hold instructions executable by the processing circuitry to implement the methods and processes described herein. When such methods and processes are implemented, the state of non-volatile storage device 306 may be transformed—e.g., to hold different data.

Non-volatile storage device 306 may include physical devices that are removable and/or built in. Non-volatile storage device 306 may include optical memory, semiconductor memory, and/or magnetic memory, or other mass storage device technology. Non-volatile storage device 306 may include nonvolatile, dynamic, static, read/write, read-only, sequential-access, location-addressable, file-addressable, and/or content-addressable devices. It will be appreciated that non-volatile storage device 306 is configured to hold instructions even when power is cut to the non-volatile storage device 306.

Volatile memory 304 may include physical devices that include random access memory. Volatile memory 304 is typically utilized by processing circuitry 302 to temporarily store information during processing of software instructions. It will be appreciated that volatile memory 304 typically does not continue to store instructions when power is cut to the volatile memory 304.

Aspects of processing circuitry 302, volatile memory 304, and non-volatile storage device 306 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 300 typically implemented in software by a processor to perform a particular function using portions of volatile memory, which function involves transformative processing that specially configures the processor to perform the function. Thus, a module, program, or engine may be instantiated via processing circuitry 302 executing instructions held by non-volatile storage device 306, using portions of volatile memory 304. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 308 may be used to present a visual representation of data held by non-volatile storage device 306. The visual representation may take the form of a graphical user interface (GUI). As the herein described methods and processes change the data held by the non-volatile storage device 306, and thus transform the state of the non-volatile storage device 306, the state of display subsystem 308 may likewise be transformed to visually represent changes in the underlying data. Display subsystem 308 may include one or more display devices utilizing virtually any type of technology. Such display devices may be combined with processing circuitry 302, volatile memory 304, and/or non-volatile storage device 306 in a shared enclosure, or such display devices may be peripheral display devices.

When included, input subsystem 310 may comprise or interface with one or more user-input devices such as a keyboard, mouse, touch screen, camera, or microphone.

When included, communication subsystem 312 may be configured to communicatively couple various computing devices described herein with each other, and with other devices. Communication subsystem 312 may include wired and/or wireless communication devices compatible with one or more different communication protocols. As non-limiting examples, the communication subsystem may be configured for communication via a wired or wireless local- or wide-area network, broadband cellular network, etc. In some embodiments, the communication subsystem may allow computing system 300 to send and/or receive messages to and/or from other devices via a network such as the Internet.

The following paragraphs discuss several aspects of the present disclosure. According to one aspect of the present disclosure, a computing system is provided, including one or more processing devices configured to, during a training phase, obtain sets of ground-state force field data associated with a respective plurality of equilibrium chemical systems. The one or more processing devices are further configured to compute respective ground-state uncertainty values of the sets of ground-state force field data. The one or more processing devices are further configured to select a first subset of the plurality of equilibrium chemical systems. The equilibrium chemical systems included in the first subset have respective ground-state uncertainty values above a ground-state uncertainty threshold. The one or more processing devices are further configured to compute a plurality of off-equilibrium chemical systems at least in part by modifying a respective temperature and/or pressure of each equilibrium chemical system included in the first subset. The one or more processing devices are further configured to compute respective ab initio simulations of a second subset of the plurality of off-equilibrium chemical systems to thereby obtain a plurality of sets of off-equilibrium force field data. The one or more processing devices are further configured to train a machine learning force fields (MLFF) model at least in part with the sets of off-equilibrium force field data. The above features may have the technical effect of generating a training dataset that allows the one or more processing devices to train the MLFF model in a sample-efficient manner. The above features may also have the technical effect of training the MLFF model to be able to accurately simulate off-equilibrium conditions.

According to this aspect, the ab initio simulations may be density functional theory (DFT) simulations. The above feature may have the technical effect of generating accurate estimates of force fields for the off-equilibrium chemical systems in the second subset.

According to this aspect, the one or more processing devices may be configured to compute the DFT simulations at a DFT estimation machine learning model. The above feature may have the technical effect of approximating DFT simulations in a more computationally efficient manner instead of performing explicit DFT simulation.

According to this aspect, for each of the equilibrium chemical systems, the one or more processing devices may be further configured to input a specification of the equilibrium chemical system into a model ensemble including a plurality of pretrained MLFF models to obtain a respective plurality of the sets of ground-state force field data. The one or more processing devices may be further configured to compute the ground-state uncertainty value for that equilibrium chemical system at least in part by computing a respective variance over the sets of ground-state force field data computed for that equilibrium chemical system at the model ensemble. The above features may have the technical effect of identifying high-uncertainty equilibrium chemical systems for which further simulation data is likely to be highly informative to the MLFF model undergoing training.

According to this aspect, the MLFF model trained during the training phase may be included in the model ensemble. The one or more processing devices may be configured to iteratively recompute the first subset of the plurality of equilibrium chemical systems during training of the MLFF model. The above features may have the technical effect of iteratively updating the uncertainty computations via active learning to increase the sample efficiency of the training corpus.

According to this aspect, the one or more processing devices may be further configured to compute a plurality of off-equilibrium uncertainty values of the off-equilibrium chemical systems. The one or more processing devices may be further configured to select, as the second subset, a plurality of the off-equilibrium chemical systems that have respective off-equilibrium uncertainty values above an off-equilibrium uncertainty threshold. The above features may have the technical effect of selecting, for ab initio simulation, off-equilibrium chemical systems that are likely to be highly informative during training of the MLFF model.

According to this aspect, the one or more processing devices may be further configured to compute a respective plurality of molecular dynamics (MD) simulations of the off-equilibrium chemical systems. The MD simulations may each include a plurality of MD snapshots. The one or more processing devices may be further configured to compute the off-equilibrium uncertainty values as uncertainty values of the MD snapshots. The above features may have the technical effect of selecting, for ab initio simulation, off-equilibrium chemical systems that have high uncertainty in their trajectories of MD snapshots.

According to this aspect, the one or more processing devices may be further configured to compute a plurality of latent space clusters that each include a respective plurality of latent space vectors computed at the MLFF model from the specifications of the equilibrium chemical systems. The one or more processing devices may be further configured to select the first subset of the plurality of equilibrium chemical systems at least in part by selecting a plurality of representative equilibrium chemical systems from the latent space clusters. The above features may have the technical effect of selecting equilibrium chemical systems that are representative of a wider portion of the overall space of equilibrium chemical systems included in the ground-state corpus.

According to another aspect of the present disclosure, a method for use with a computing system is provided. The method includes, during a training phase, obtaining sets of ground-state force field data associated with a respective plurality of equilibrium chemical systems. The method further includes computing respective ground-state uncertainty values of the sets of ground-state force field data. The method further includes selecting a first subset of the plurality of equilibrium chemical systems. The equilibrium chemical systems included in the first subset have respective ground-state uncertainty values above a ground-state uncertainty threshold. The method further includes computing a plurality of off-equilibrium chemical systems at least in part by modifying a respective temperature and/or pressure of each equilibrium chemical system included in the first subset. The method further includes computing respective ab initio simulations of a second subset of the plurality of off-equilibrium chemical systems to thereby obtain a plurality of sets of off-equilibrium force field data. The method further includes training a machine learning force fields (MLFF) model at least in part with the sets of off-equilibrium force field data. The above features may have the technical effect of generating a training dataset that allows the one or more processing devices to train the MLFF model in a sample-efficient manner. The above features may also have the technical effect of training the MLFF model to be able to accurately simulate off-equilibrium conditions.

According to this aspect, the ab initio simulations may be density functional theory (DFT) simulations. The above feature may have the technical effect of generating accurate estimates of force fields for the off-equilibrium chemical systems in the second subset.

According to this aspect, the DFT simulations may be computed at a DFT estimation machine learning model. The above feature may have the technical effect of approximating DFT simulations in a more computationally efficient manner instead of performing explicit DFT simulation.

According to this aspect, the method may further include, for each of the equilibrium chemical systems, inputting a specification of the equilibrium chemical system into a model ensemble including a plurality of pretrained MLFF models to obtain a respective plurality of the sets of ground-state force field data. The method may further include computing the ground-state uncertainty value for that equilibrium chemical system at least in part by computing a respective variance over the sets of ground-state force field data computed for that equilibrium chemical system at the model ensemble. The above features may have the technical effect of identifying high-uncertainty equilibrium chemical systems for which further simulation data is likely to be highly informative to the MLFF model undergoing training.

According to this aspect, the MLFF model trained during the training phase may be included in the model ensemble. The method may further include iteratively recomputing the first subset of the plurality of equilibrium chemical systems during training of the MLFF model. The above features may have the technical effect of iteratively updating the uncertainty computations via active learning to increase the sample efficiency of the training corpus.

According to this aspect, the method may further include computing a plurality of off-equilibrium uncertainty values of the off-equilibrium chemical systems. The method may further include selecting, as the second subset, a plurality of the off-equilibrium chemical systems that have respective off-equilibrium uncertainty values above an off-equilibrium uncertainty threshold. The above features may have the technical effect of selecting, for ab initio simulation, off-equilibrium chemical systems that are likely to be highly informative during training of the MLFF model.

According to this aspect, the method may further include computing a plurality of latent space clusters that each include a respective plurality of latent space vectors computed at the MLFF model from the specifications of the equilibrium chemical systems. The method may further include selecting the first subset of the plurality of equilibrium chemical systems at least in part by selecting a plurality of representative equilibrium chemical systems from the latent space clusters. The above features may have the technical effect of selecting equilibrium chemical systems that are representative of a wider portion of the overall space of equilibrium chemical systems included in the ground-state corpus.

According to another aspect of the present disclosure, a computing system is provided, including one or more processing devices configured to, during an inferencing phase, at a trained machine learning force fields (MLFF) model, compute a set of inferencing-time force field data respectively associated with an inferencing-time chemical system. The one or more processing devices are further configured to output the set of inferencing-time force field data. The trained MLFF model is trained at least in part by, during a training phase, obtaining sets of ground-state force field data associated with a respective plurality of equilibrium chemical systems. Training the MLFF model further includes computing respective ground-state uncertainty values of the sets of ground-state force field data. Training the MLFF model further includes selecting a first subset of the plurality of equilibrium chemical systems. The equilibrium chemical systems included in the first subset have respective ground-state uncertainty values above a ground-state uncertainty threshold. Training the MLFF model further includes computing a plurality of off-equilibrium chemical systems at least in part by modifying a respective temperature and/or pressure of each equilibrium chemical system included in the first subset. Training the MLFF model further includes computing respective ab initio simulations of a second subset of the plurality of off-equilibrium chemical systems to thereby obtain a plurality of sets of off-equilibrium force field data. The MLFF model is trained at least in part with the sets of off-equilibrium force field data. The above features may have the technical effect of generating a training dataset that allows the one or more processing devices to train the MLFF model in a sample-efficient manner. The above features may also have the technical effect of accurately simulating off-equilibrium conditions at the trained MLFF model.

According to this aspect, the one or more processing devices may be further configured to, based at least in part on the inferencing-time force field data, predict whether the inferencing-time chemical system is stable. The one or more processing devices may be further configured to output the prediction of whether the inferencing-time chemical system is stable. The above features may have the technical effect of identifying stable materials using the trained MLFF model.

According to this aspect, the one or more processing devices may be configured to predict whether a plurality of inferencing-time chemical systems are stable at least in part by estimating respective formation energy values of the inferencing-time chemical systems. Predicting whether the inferencing-time chemical systems are stable may further include iteratively recomputing a convex hull of the plurality of formation energy values. The one or more processing devices may be further configured to determine that at least one of the inferencing-time chemical systems is stable in response to determining that the at least one inferencing-time chemical system has a respective formation energy value on or below the convex hull. The above features may have the technical effect of incorporating previous stability determinations when determining whether an inferencing-time chemical system is stable.

According to this aspect, the one or more processing devices may be further configured to, based at least in part on the inferencing-time force field data, predict a phonon dispersion in the inferencing-time chemical system. The one or more processing devices may be further configured to output the phonon dispersion. The above features may have the technical effect of predicting phonon properties of the inferencing-time chemical system.

According to this aspect, the one or more processing devices may be further configured to, based at least in part on the inferencing-time force field data, compute molecular dynamics simulation data of the inferencing-time chemical system. The one or more processing devices may be further configured to output the molecular dynamics simulation data. The above features may have the technical effect of predicting molecular dynamics of the inferencing-time chemical system.

“And/or” as used herein is defined as the inclusive or V, as specified by the following truth table:

| A | B | A ∨ B |
|---|---|---|
| True | True | True |
| True | False | True |
| False | True | True |
| False | False | False |

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system comprising:
one or more processing devices configured to, during a training phase:
obtain sets of ground-state force field data associated with a respective plurality of equilibrium chemical systems;
compute respective ground-state uncertainty values of the sets of ground-state force field data;
select a first subset of the plurality of equilibrium chemical systems, wherein the equilibrium chemical systems included in the first subset have respective ground-state uncertainty values above a ground-state uncertainty threshold;
compute a plurality of off-equilibrium chemical systems at least in part by modifying a respective temperature and/or pressure of each equilibrium chemical system included in the first subset;
compute respective ab initio simulations of a second subset of the plurality of off-equilibrium chemical systems to thereby obtain a plurality of sets of off-equilibrium force field data; and
train a machine learning force fields (MLFF) model at least in part with the sets of off-equilibrium force field data.

2. The computing system of claim 1, wherein the ab initio simulations are density functional theory (DFT) simulations.

3. The computing system of claim 2, wherein the one or more processing devices are configured to compute the DFT simulations at a DFT estimation machine learning model.

4. The computing system of claim 1, wherein, for each of the equilibrium chemical systems, the one or more processing devices are configured to:
input a specification of the equilibrium chemical system into a model ensemble including a plurality of pretrained MLFF models to obtain a respective plurality of the sets of ground-state force field data; and
compute the ground-state uncertainty value for that equilibrium chemical system at least in part by computing a respective variance over the sets of ground-state force field data computed for that equilibrium chemical system at the model ensemble.

5. The computing system of claim 4, wherein:
the MLFF model trained during the training phase is included in the model ensemble; and
the one or more processing devices are configured to iteratively recompute the first subset of the plurality of equilibrium chemical systems during training of the MLFF model.

6. The computing system of claim 1, wherein the one or more processing devices are further configured to:
compute a plurality of off-equilibrium uncertainty values of the off-equilibrium chemical systems; and
select, as the second subset, a plurality of the off-equilibrium chemical systems that have respective off-equilibrium uncertainty values above an off-equilibrium uncertainty threshold.

7. The computing system of claim 6, wherein the one or more processing devices are further configured to:
compute a respective plurality of molecular dynamics (MD) simulations of the off-equilibrium chemical systems, wherein the MD simulations each include a plurality of MD snapshots; and
compute the off-equilibrium uncertainty values as uncertainty values of the MD snapshots.

8. The computing system of claim 1, wherein the one or more processing devices are further configured to:
compute a plurality of latent space clusters that each include a respective plurality of latent space vectors computed at the MLFF model from the specifications of the equilibrium chemical systems; and
select the first subset of the plurality of equilibrium chemical systems at least in part by selecting a plurality of representative equilibrium chemical systems from the latent space clusters.

9. A method for use with a computing system, the method comprising, during a training phase:
obtaining sets of ground-state force field data associated with a respective plurality of equilibrium chemical systems;
computing respective ground-state uncertainty values of the sets of ground-state force field data;
selecting a first subset of the plurality of equilibrium chemical systems, wherein the equilibrium chemical systems included in the first subset have respective ground-state uncertainty values above a ground-state uncertainty threshold;
computing a plurality of off-equilibrium chemical systems at least in part by modifying a respective temperature and/or pressure of each equilibrium chemical system included in the first subset;
computing respective ab initio simulations of a second subset of the plurality of off-equilibrium chemical systems to thereby obtain a plurality of sets of off-equilibrium force field data; and
training a machine learning force fields (MLFF) model at least in part with the sets of off-equilibrium force field data.

10. The method of claim 9, wherein the ab initio simulations are density functional theory (DFT) simulations.

11. The method of claim 10, wherein the DFT simulations are computed at a DFT estimation machine learning model.

12. The method of claim 9, further comprising, for each of the equilibrium chemical systems:
inputting a specification of the equilibrium chemical system into a model ensemble including a plurality of pretrained MLFF models to obtain a respective plurality of the sets of ground-state force field data; and
computing the ground-state uncertainty value for that equilibrium chemical system at least in part by computing a respective variance over the sets of ground-state force field data computed for that equilibrium chemical system at the model ensemble.

13. The method of claim 12, wherein:
the MLFF model trained during the training phase is included in the model ensemble; and
the method further comprises iteratively recomputing the first subset of the plurality of equilibrium chemical systems during training of the MLFF model.

14. The method of claim 9, further comprising:
computing a plurality of off-equilibrium uncertainty values of the off-equilibrium chemical systems; and
selecting, as the second subset, a plurality of the off-equilibrium chemical systems that have respective off-equilibrium uncertainty values above an off-equilibrium uncertainty threshold.

15. The method of claim 9, further comprising:
computing a plurality of latent space clusters that each include a respective plurality of latent space vectors computed at the MLFF model from the specifications of the equilibrium chemical systems; and
selecting the first subset of the plurality of equilibrium chemical systems at least in part by selecting a plurality of representative equilibrium chemical systems from the latent space clusters.

16. A computing system comprising:
one or more processing devices configured to, during an inferencing phase:
at a trained machine learning force fields (MLFF) model, compute a set of inferencing-time force field data respectively associated with an inferencing-time chemical system; and
output the set of inferencing-time force field data, wherein the trained MLFF model is trained at least in part by, during a training phase:
obtaining sets of ground-state force field data associated with a respective plurality of equilibrium chemical systems;
computing respective ground-state uncertainty values of the sets of ground-state force field data;
selecting a first subset of the plurality of equilibrium chemical systems, wherein the equilibrium chemical systems included in the first subset have respective ground-state uncertainty values above a ground-state uncertainty threshold;
computing a plurality of off-equilibrium chemical systems at least in part by modifying a respective temperature and/or pressure of each equilibrium chemical system included in the first subset;
computing respective ab initio simulations of a second subset of the plurality of off-equilibrium chemical systems to thereby obtain a plurality of sets of off-equilibrium force field data; and
training the MLFF model at least in part with the sets of off-equilibrium force field data.

17. The computing system of claim 16, wherein the one or more processing devices are further configured to:
based at least in part on the inferencing-time force field data, predict whether the inferencing-time chemical system is stable; and
output the prediction of whether the inferencing-time chemical system is stable.

18. The computing system of claim 17, wherein the one or more processing devices are configured to predict whether a plurality of inferencing-time chemical systems are stable at least in part by:
estimating respective formation energy values of the inferencing-time chemical systems;
iteratively recomputing a convex hull of the plurality of formation energy values; and
determining that at least one of the inferencing-time chemical systems is stable in response to determining that the at least one inferencing-time chemical system has a respective formation energy value on or below the convex hull.

19. The computing system of claim 16, wherein the one or more processing devices are further configured to:
based at least in part on the inferencing-time force field data, predict a phonon dispersion in the inferencing-time chemical system; and
output the phonon dispersion.

20. The computing system of claim 16, wherein the one or more processing devices are further configured to:
based at least in part on the inferencing-time force field data, compute molecular dynamics simulation data of the inferencing-time chemical system; and
output the molecular dynamics simulation data.
